(19) United States
(12) Patent Application Publication
(10) Pub. No.: US 2025/0364139 A1
(43) Pub. Date: Nov. 27, 2025

(54) TUMOR MARKER SELECTION AND DETECTION

(71) Applicant: SAGA Dx, Inc., Morrisville, NC (US)

(72) Inventors: Miguel Alcaide, Lund (SE); Anthony Miles George, Lund (SE); Roopom Banerjee, Morrisville, NC (US); Samuel Woodhouse, Lund (SE)

(21) Appl. No.: 19/213,736

(22) Filed: May 20, 2025

Related U.S. Application Data
(60) Provisional application No. 63/650,052, filed on May 21, 2024.

(51) Int. Cl. G16H 50/20; C12Q 1/6809; C12Q 1/6869; C12Q 1/6886; G16B 20/20; G16B 25/20; G16H 15/00

(52) U.S. Cl. CPC G16H 50/20; C12Q 1/6809; C12Q 1/6869; C12Q 1/6886; G16B 20/20; G16B 25/20; G16H 15/00

(57) ABSTRACT

Methods for detecting a tumor using a sample in which tumor DNA fragments are present only in a very low concentration, beyond the statistical limit of detection, where methods include: obtaining sequence data for tumor nucleic acid from a tumor from a subject and analyzing the sequence data to identify a plurality of tumor-specific variants that are in the tumor nucleic acid and that are not in non-tumor nucleic acid of the subject; selecting a marker variant that appears duplicated in tumor nucleic acid (compared to non-tumor nucleic acid) a greater number of times than other ones of the tumor variants; performing an assay to detect the marker variant in a sample from the subject; and reporting the presence of the tumor in the subject when the assay is positive for the marker variant in the sample.

### TECHNICAL FIELD

The disclosure relates to identifying and detecting variants as biomarkers of diseases.

### BACKGROUND

Circulating cell-free tumor DNA (ctDNA) is a promising biomarker for cancer detection. Cell-free DNA (cfDNA), including ctDNA, is released from cells into various body fluids, most importantly circulating blood. A significant challenge in the use of ctDNA for liquid biopsy (e.g., blood or plasma samples) is the low number of target ctDNA molecules in blood.

Literature suggests that, among cell-free DNA circulating in plasma, circulating tumor DNA (ctDNA) is present in an amount on the order of one copy per 1000 cfDNA copies (0.1%), potentially increasing to about 0.3% during tumor progression and reaching only about 10% at metastasis. See Roberto, 2023, Strategies for improving detection of circulating tumor DNA using next generation sequencing, Canc Treatment Rev 119:102595, incorporated by reference. The implication is that tumor detection assays should have low limits of detection (LoD), lower than 0.1% variant allele frequency (VAF). It is estimated that at least 3.3 ng of cfDNA is needed to have at least 1 copy of a mutation at 0.1% in plasma. Individuals rarely have more than 30 ng cfDNA per ml of plasma, with most having less than 10 ng per ml plasma. See Johansson, 2019, Considerations and quality controls when analyzing cell-free tumor DNA, Biomol Detec Quant 17:100078, incorporated by reference.

Unfortunately, the chance of detecting ctDNA in plasma is worse than theoretical calculations predict due to a number of other factors. For example, cfDNA is highly fragmented and there is a probability that the target of interest is broken into fragments too small to detect. Detection methods lead to loss of the DNA during processing. Also, clinical sampling is subject to stochastic phenomena. Simply put, there is a good chance a target molecule of-interest will avoid being included in a sample or lost in the “dead volume” of lab reactions.

### SUMMARY

The present invention provides methods for the detection of tumors and evidence of minimal residual disease (MRD). In methods of the disclosure, tumor nucleic acid, from a tumor sample, is sequenced and the sequence data are analyzed to identify tumor-specific variants that constitute a tumor mutation profile or tumor signature. Each variant is found specifically in tumor nucleic acid and is not present in healthy, non-tumor nucleic acid from the subject. In that sense, the variants are specific to the tumor and may be referred to as tumor variants. Once the tumor variants are identified, methods include selecting one or more of the tumor variants that are present in the tumor nucleic acid at high copy number. Selected variants are used as tumor biomarkers (alternatively, a marker variant because it may be used subsequently as a biomarker for the presence of the tumor in the subject). The marker variant is selected because that variant appears and has been duplicated in the tumor nucleic acid, compared to non-tumor nucleic acid, a greater number of times than other tumor variants. That is, some of the tumor variants may appear once in the tumor nucleic acid and not in non-tumor nucleic acid obtained from a subject, but the marker variant appears a greater number of times, such as five, or two, or seven, or dozens. After the marker variant is selected, the invention provides amplification reagents such as a primer pair specific for the marker variant. That primer pair may be used subsequently to assay for the marker variant in a sample from a subject as evidence of the presence of the tumor, e.g., in an assay for MRD after the subject has undergone a treatment to eradicate the tumor.

In one aspect of the invention, the tumor DNA is extrachromosomal DNA (ecDNA), which is a class of tumor nucleic acid that is not integrated in chromosomes. Originating from the tumor genome, ecDNA often includes amplified oncogenes or tumor-specific variants, rendering it a source of biomarkers for detecting and characterizing cancer.

Because the marker variant has been selected for increased copy number-compared to other genomic loci in the tumor nucleic acid-the marker variant is present among circulating tumor DNA (e.g., ecDNA) in amplified quantities relative to ctDNA harboring any single loci that has not undergone copy number amplification. Selecting the marker variant for having an amplified copy number in the tumor nucleic acid exploits a form of in vivo pre-amplification in which a tumor-specific sequence is increased in abundance relative to tumor DNA and possibly by mechanisms associated with the oncogenic phenomena that gave rise to the tumor. That is, methods of the invention involve selecting a target (marker variant) that has been amplified in copy number, relative to other tumor variants, in vivo by mechanisms occurring in the tumor cells or their progenitor cells. One such mechanism involves the incorporation of the marker variant into extrachromosomal DNA (ecDNA). The ecDNA is circular, can replicate with a degree of autonomy from chromosomal DNA, and accumulate to very high copy numbers (e.g., tens to hundreds of copies per cell), thereby increasing the abundance of the marker variant they carry. Because the marker variant has undergone in vivo pre-amplification, that variant is similarly amplified, i.e., superabundant, among ctDNA. By selecting such an amplified target, methods of the invention allow tumor nucleic acid to be detected with a sensitivity that breaks past the statistical limits conventionally associated with the chance of detecting any given ctDNA in a sample such as plasma. For example, for assays such as liquid biopsy where, due to sample size, stochastic sampling, or dead volumes, a target is expected to go undetected 80% of the time, where methods of the invention detect a variant that has been amplified to five copies in a chromosome, then the selection of the copy number amplified gene as the marker variant allows the assay to break the statistical limit of detection (LoD). Even though any single (unduplicated) locus in the tumor DNA would be beyond the LoD for an assay, an assay of the invention is mathematically and biologically favored to detect the marker variant and to thus show evidence of the presence of the tumor in the subject.

Because selecting a high copy number tumor variant as a marker variant provides for a tumor assay that beats the LoD that would be associated with other conditions of the assay (sample size, nature of ctDNA, VAF, etc.), assays of the invention are very sensitive and useful for the detection of minimal residual disease (MRD). Preferred embodiments provide methods that fit within a two-stage workflow. At a first stage, tumor material (e.g., from tissue biopsy or liquid biopsy), is provided and tumor nucleic acid is sequenced to obtain sequence data. The tumor material may be obtained from any body fluid, including but not limited to plasma, cerebral spinal fluid, and the like. The sequence data are analyzed to discover a plurality of tumor variants, generally “structural variants” or tumor SVs (although polymorphisms and small indels are within the scope of the disclosure). For each of the tumor SVs, copy number is estimated, and the tumor SVs may be ranked by copy number (higher copy number corresponding to a higher rank). At least one high ranking, or high copy number SV, is deemed to be a marker variant and a second stage assay is designed and/or performed to detect that marker variant in a sample. In preferred embodiments, the second stage detection assay is digital PCR, and methods include designing a primer pair that specifically and exclusively amplifies the marker variant. The detection assay may be multiplex in nature and probe for several tumor SVs simultaneously. The detection assay, such as a digital PCR assay on a biological sample (e.g., a blood or plasma sample, CSF, a pleural effusion, urine, saliva and the like) obtained from the subject after treatment, has the ability to detect evidence of the tumor even when ctDNA is present at only a very small VAF. Thus, the assay may be performed after treatment to detect any evidence of MRD or tumor recurrence. In certain embodiments, the copy number and presence of SVs are confirmed by orthogonal validation and the highest ranked SVs are selected.

The source of the tumor material can be a (or is suspected of being a) diseased cell, fluid, tissue, or organ. For example, the source of a sample can be an individual who may or may not have cancer—and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. The sample can be a cell-free liquid sample or a liquid sample that comprises cells.

In certain aspects, the invention provides methods for detecting a tumor using a sample in which tumor DNA fragments are present only in a very low concentration, beyond the statistical limit of detection. The methods include obtaining sequence data for tumor nucleic acid from a tumor from a subject and analyzing the sequence data to identify a plurality of tumor-specific variants that are in the tumor nucleic acid and that are not in non-tumor nucleic acid of the subject. A marker variant that appears duplicated in tumor nucleic acid (compared to non-tumor nucleic acid) a greater number of times than other ones of the plurality of tumor-specific variants is selected. The method includes performing an assay to detect the marker variant in a sample from the subject and reporting the presence of the tumor in the subject when the assay is positive for the marker variant in the sample. The assay may use an amplification reaction under conditions at which an unduplicated locus in the tumor nucleic acid would be statistically beyond a limit of detection. In some embodiments, the sample includes blood or plasma from the subject and the assay comprises digital PCR to detect cell-free nucleic acid in the blood or plasma. For example, in digital PCR with liquid biopsy, the sample may include cell-free DNA from blood or plasma and the assay may involve dividing the sample into a plurality of aqueous partitions such that at least one partition includes one fragment of the cell-free DNA that includes one copy of the marker variant that was duplicated within the tumor nucleic acid. For digital PCR embodiments, the method may include providing each of the plurality of aqueous partitions with PCR reagents, a primer pair useful to amplify the variant, and detectably labeled probes for an amplification product of the primer pair. Due to a quantity of the cell-free DNA circulating in the blood or plasma in the subject and due to a volume of the sample, it may be mathematically more probable that (i) none of the aqueous partitions would contain any copy of an unduplicated locus in the tumor nucleic acid than that (ii) any copy of the unduplicated locus would appear among the aqueous partitions.

The sequence data may be obtained by sequencing DNA from a source of tumor material (e.g., from a biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) from the subject to obtain sequence reads. The tumor material may be obtained from a tissue biopsy, or other biological sample, such as blood, urine, stool, or cerebrospinal fluid from the patient. The tumor material may further be formalin-fixed paraffin-embedded or fresh frozen. In some embodiments, the sequencing uses low-pass, whole genome sequencing (LP-WGS) protocol. The analyzing step may include mapping the sequence reads to a reference genomic sequence and identifying read mappings that indicate a structural variant in the tumor nucleic acid relative to the non-tumor nucleic acid of the subject. The method may further include identifying additional features of the structural variants, such as the variant type (e.g., inversion, tandem duplication, inter-chromosomal), clonal prevalence (present in all tumor cells or only a subset of tumor cells), or inclusion or association with an ecDNA construct. The method may include designing a primer pair useful to amplify the marker variant, when the assay comprises an amplification reaction.

In certain embodiments, the sequence data is obtained by sequencing DNA from an FFPE slice of the tumor; the subject has undergone treatment to eradicate the tumor; the aqueous partitions are aqueous droplets; the assay is droplet digital PCR; the detectably labeled probes are fluorescent hydrolysis probes; detecting fluorescence from the aqueous droplets indicates the presence of the tumor nucleic acid in the sample; and/or the assay is performed to detect minimal residual disease after the treatment.

Aspects provide a tumor detection test/assay and related methods that are compatible with liquid biopsy or blood draw and may be used to detect a tumor in a patient from cell-free nucleic acid that is present at a very low concentration such that any given target or locus is statistically likely to be beyond/below the limit of detection. Such methods for detecting indicia of disease may include identifying a sequence that is duplicated within tumor nucleic acid from a tumor compared to non-tumor nucleic acid of the subject and performing an assay to detect the sequence in a sample from the subject at conditions under which an unduplicated genomic locus of the subject is more likely to be undetectable than to be detectable. When the sequence is detected using the assay, the method includes reporting the presence of the tumor in the subject.

Considering the low concentration of tumor cfDNA relative to the analyzed sample volume, the probability of capturing a single copy of an unduplicated locus or non-ecDNA elements in a partition may be lower than the probability of capturing no copies of the unduplicated locus or non-ecDNA elements6. This makes consistent detection of such low-frequency, single-copy markers difficult.

The sample may include blood or plasma from the subject and method may include capturing cell-free nucleic acid from the blood or plasma and performing the assay on the cell-free nucleic acid.

In certain digital PCR embodiments, the assay includes partitioning the sample into a plurality of discrete reaction volumes. These partitions can be, for example, aqueous droplets within a non-aqueous emulsion (e.g., in droplet-based dPCR systems) or defined reaction chambers or wells on a solid substrate (e.g., plate-based or chip-based dPCR systems, e.g., QIAcuity). An amplification reaction is subsequently performed within these individual partitions using at least one primer specific for the target sequence and a probe that provides a detectable signal when the amplification reaction generates an amplification product.

For droplet digital PCR (ddPCR), the aqueous partitions may be aqueous droplets and the assay comprises performing the ddPCR with sequence-specific fluorescent hybridization probes. In some embodiments, the assay is for cell-free nucleic acid, wherein the sample is less than about 100 mL of blood or plasma, and the cell-free nucleic acid is present at a concentration between about 1 and 50 ng/ml or lower in the blood or plasma circulating in the subject, and the targeted sequence is duplicated to at least about five copies in a genome of the tumor.

For formalin-fixed, paraffin embedded (FFPE) embodiments, the identifying step comprises sequencing DNA from an FFPE slice of the tumor to obtain sequence reads, mapping the sequence reads to a reference, and identifying read-mappings consistent with a structural variant that is duplicated in the tumor nucleic acid. The method may also perform an additional step to characterize the presence or absence of ecDNA in the sample. Preferably, the identifying step comprises sequencing DNA from the tumor to obtain sequence reads, mapping the reads to a reference to identify a plurality of tumor-specific structural variants (SVs), ranking the SVs by copy number wherein higher ranks are correlated to higher copy numbers, and selecting a high-ranking SV as the sequence. The assay may be designed to detect two or more of the SVs with higher ranks as a patient-specific, tumor-specific signature of the tumor in the subject. The method may include designing and providing a plurality of copies of a primer pair that specifically amplify the sequence and storing the plurality of copies of a primer pair as reagents for use in one or more future assays for minimal residual disease.

Aspects of the invention provide methods for detecting tumors that take advantage of in vivo preamplification of certain tumor targets. An example of such preamplification is the focal amplification of specific genomic regions, e.g., oncogenes, which can arise from their incorporation into and subsequent replication as extra-chromosomal DNA (ecDNA) elements within tumor cells. These ecDNA elements can accumulate to high copy numbers, leading to a significant in vivo increase in the abundance of the target sequences they carry.

Methods may be used in assays for minimal residual disease (MRD), e.g., after a treatment to eradicate the tumor. Methods my include obtaining a sample from a subject who has undergone treatment for a tumor; performing an amplification reaction in the sample using a primer pair that is designed to amplify a tumor-specific marker variant that appears duplicated in tumor nucleic acid from the tumor, compared to non-tumor nucleic acid from the subject, a greater number of times than other tumor-specific variants that have been shown to be present in the tumor nucleic acid; and reporting the residual presence of the tumor after the treatment when the primer pair generates amplicons by the amplification reaction. The sample may be obtained by receiving a blood collection tube or container containing blood or plasma that was obtained from the subject via blood draw. The sample may include cell-free DNA from blood or plasma from the subject. In some embodiments, the sample is less than about 100 mL of blood or plasma and the cell-free DNA is present at a concentration between about 1 and 50 ng/ml or lower in the blood or plasma circulating in the subject. The detection methods are effective beyond the LoD that would limit conventional MRD tests relying on ctDNA in a liquid biopsy sample. For example, it may be that under conditions of the amplification reaction it is more probable that an unduplicated genomic locus from the tumor would not encounter the primer pair than that the unduplicated genomic locus would encounter the primer pair. The method may include partitioning the sample into aqueous partitions that include PCR reagents and fluorescent probes for the amplicons and conducting the amplification reaction in the aqueous partitions. The method may include detecting fluorescence from the partitions to detect the residual presence of the tumor after the treatment.

In certain embodiments, the amplification reaction uses a plurality of primer pairs designed to amplify a respective plurality of structural variants (SVs), wherein one or more SV of the plurality of SVs has been shown to exhibit copy amplification in the tumor nucleic acid compared to the non-tumor nucleic acid from the subject. The plurality of primer pairs may be provided as a reagent in one or more containers for use in the amplification reaction for detection of the plurality of SVs as a tumor-specific, patient specific signature of presences of the tumor. The plurality of SVs may be detected in multiplex in the one amplification reaction using a respective plurality of detectably labeled probes.

In some aspects, the invention provides methods for ranking structural variants (SVs) and/or otherwise detecting and assigning relative ranks, in terms of clinical diagnostic utility, to a plurality of tumor-specific biomarkers, such as tumor-specific variants in tumor nucleic acid. In certain embodiments, the ranking is informed by factors that include, but are not limited to, the biomarker's association with extrachromosomal DNA (ecDNA), established or predicted linkage to therapeutic response or resistance, or impact on or proximity to genomic regions relevant to therapeutic targets.

Systematically ranking SVs provides an approach for the automatic selection of which SVs to interrogate in a diagnostic assay, such as a digital PCR assay for circulating-tumor DNA in blood or plasma. Methods include analyzing sequence data from tumor nucleic acid from a tumor of a subject to identify the presence and copy numbers of a plurality of tumor-specific structural variants (SVs) in the tumor nucleic acid compared to non-tumor nucleic acid from the subject; ranking the SVs, wherein higher ranks are correlated to higher copy numbers (or vice-versa); and providing reagents for an assay that detects a tumor signature comprising one or more of the SVs selected for having the higher ranks (or lower, i.e., having a rank indicating a relatively higher copy number). The reagents may include primer pairs that amplify copies of the one or more SVs. The method may further include performing the assay on a sample from a subject to detect the tumor in the subject by detecting copies of the one or more SVs. For example, the copies may be detected in cell free DNA from blood or plasma in the sample. The assay may be amplification-based such as, for example, digital PCR to detect amplification of the copies of the one or more SVs. The sample may include less than about 100 mL of the blood or plasma and wherein the cell-free DNA is present at a concentration between about 1 and 50 ng/mL, or lower, in the blood or plasma circulating in the subject. In some embodiments, under conditions of the amplification reaction it is more probable that an unduplicated genomic locus from the tumor would not be present in the sample than that the unduplicated genomic locus would be present in the sample such that by ranking the variants and using the ranking to select high copy number variants for the assay, the detection assay detects evidence of the tumor.

The ranking step may further include assigning a high rank to a truncal SV identified as an initiating truncal mutation of the tumor. The process may also involve adjusting ranks based on other characteristics; e.g., consideration may be given to an SV's presence on, or association with, extrachromosomal DNA (ecDNA). Further, SVs may be selectively prioritized (i.e., up-ranked) or deprioritized (i.e., down-ranked) based on the known disease linkage or clinical significance of the SV itself, or of any genes whose function, structure, or copy number is altered by the SV (e.g., known oncogenes or tumor suppressors associated with the disease).

The analyzing step includes analyzing sequence data from the tumor at multiple different times to identify a persistent SV present in the tumor at the different times and assigning a top rank to the persistent SV. The ranking step may be implemented informatically and may be performed by a computer system that is analyzing the sequence data from the tumor nucleic acid from a tumor of a subject to identify the presence and copy numbers of the plurality of tumor-specific structural variants (SVs). The computer system may be programmed to automatically rank SVs for each sample analyzed, e.g., as part of the workflow for identifying the SVs.

In digital PCR embodiments, the method may include partitioning the sample into aqueous partitions that include PCR reagents and fluorescent probes for the amplicons; conducting the amplification reaction in the aqueous partitions; and detecting fluorescence from the partitions to detect the residual presence of the tumor after the treatment.

### DETAILED DESCRIPTION

The invention provides methods that are useful to detect a tumor in a subject using a detection assay at conditions under which any given tumor genomic locus is beyond the statistical limit of detection (LoD). A conventional blood draw for liquid biopsy may collect about 10 mL of blood or plasma in a 10 mL blood collection tube. One may typically find about 30 ng cell-free DNA (cfDNA) or less per mL of plasma. In an early stage but diagnosable tumor, any given circulating tumor DNA (ctDNA) fragment may be present at about one copy per 1000 cfDNA copies (0.1%), and much lower after treatment when the concern is minimal residual disease (MRD). Due to those numbers, literature suggests that it is statistically improbably to detect any given ctDNA fragment from a conventional liquid biopsy blood draw and it has been suggested much greater volumes of blood would be required to achieve good (e.g., 95%) sensitivity. See Connal, 2023, Liquid biopsies: the future of cancer early detection, J Transl Med 21:118, incorporated by reference. Methods of the invention break that statistical LoD using an analytical workflow that includes analyzing sequence data from tumor nucleic acid to identify a plurality of tumor-specific variants, and then selecting one of those identified tumor variants that has an amplified copy number, in the tumor nucleic acid, relative to the others, and that is not found in healthy, non-tumor DNA of the subject. That amplified copy number tumor variant may be deemed a marker variant for purposes herein and an assay is prepared and/or performed to detect the marker variant in a sample from the subject as a test for evidence of the presence of the tumor in the subject.

The initial sequence data analysis, which may involve next-generation sequencing (NGS) of a tumor sample such as from a biopsy or a formalin-fixed, paraffin embedded (FFPE) tumor slice, and may proceed by low-pass, whole genome sequencing (LP-WGS), may be performed at one point in time to detect the plurality of tumor-specific variants. Those variants are analyzed for copy number amplification, aka duplication, i.e., structural variants (SVs) in which genomic segments exhibit duplication. Methods of the invention may include ranking the SVs by copy number. The ranking may be implemented informatically, performed automatically by a computer system executing software instructions such as a bioinformatics pipeline. Preferably, the SVs are assigned relative ranks correlating to their respective copy number in the tumor nucleic acid. Then, methods of the invention may involve selecting, based on rank, one high copy number SV or a panel of such SVs to be used as a tumor signature, to be probed for in a detection assay such as a digital PCR assay with a liquid biopsy sample as a test for MRD after treatment.

By those methods, the invention provides methods for detecting a tumor using a sample in which any single tumor DNA fragments are present only in a very low concentration, beyond the statistical LoD. The detection assay breaks the statistical LoD at least in that, due to a quantity of the cell-free DNA circulating in the blood or plasma in the subject and due to a volume of the sample, it is mathematically more probable that (i) none of the partitions would contain any copy of an unduplicated locus in the tumor nucleic acid than that (ii) any copy of the unduplicated locus would appear among the aqueous partitions. Assays for detecting unduplicated, tumor-specific loci in cell-free DNA (cfDNA) face limitations from the statistical limit of detection (LoD). This limitation is evident given the low quantity of tumor-derived cfDNA circulating in a subject's biological sample (e.g., blood or plasma) relative to the sample volume processed. In such scenarios, when analyzing the sample through discrete partitions (e.g., droplets in digital PCR or reads in a sequencing run), it can be mathematically more probable that any given partition will contain zero copies of the unduplicated tumor locus than it is to contain one or more copies. These partitions may be solid or aqueous.

Rather than interrogating the sample for an unduplicated locus, methods of the invention exploit a form of in vivo pre-amplification whereby the marker variant has been amplified, i.e., duplicated, in the tumor genome relative to other loci. In various embodiments described herein, the sequence data may be obtained by sequencing DNA from an FFPE slice of the tumor; a library preparation protocol tailored to FFPE-sourced nucleic acid may be used; the tumor nucleic acid may be sequenced by LP-WGS; a computer system may be used to detect and rank tumor SVs and select a marker variant and to design primers specific for the marker variant; the primer pair may be used in a detection assay for a subject that has undergone treatment to eradicate the tumor; the sample may be a blood draw liquid biopsy, the detection assay may involve digital PCR with the sample in aqueous partitions using an amplification reaction and fluorescent hydrolysis probes; detecting fluorescence from the aqueous droplets may indicate the presence of the tumor nucleic acid in the sample; and/or the assay may be performed to detect minimal residual disease after the treatment.

### FFPE DNA Extraction

Methods of the disclosure may include obtaining nucleic acid from a formalin-fixed, paraffin embedded slice of a tumor, so that the tumor nucleic acid may be sequenced. Tissue obtained by biopsy or surgery for pathological examination may be fixed in a fixative, such as formalin and embedded in paraffin, yielding formalin fixed, paraffin embedded (FFPE) blocks. Small (e.g., a few micrometer-thick) sections may be sliced from the blocks and stained on slides for microscopic analysis. Such slides are typically retained as a pathology archive.

Methods herein may use protocols for extracting DNA from FFPE samples and preparing high-quality sequencing libraries from the FFPE-extracted DNA. To extract nucleic acid, the sample is loaded into a tube such as microcentrifuge tube. A tissue lysis buffer and proteinase K (PK) solution mix may be added to the tube. Steps of protocols herein may be performed using reagents and material sold under the product name truXTRAC FFPE total NA (tNA) Ultra Kit by Covaris. The FFPE sample may be immersed in the tissue lysis buffer/PK solution mix and sonicated in a ultrasonication instrument according to manufacturer instructions for paraffin emulsification. The steps may be performed in laboratory test tubes, wells of a plate, microcentrifuge tubes, or tubes in a multi-tube strip.

After the tube is collected, it is centrifuged, e.g., spun at 5 k g for about 15 minutes, to form a pellet that includes DNA. The described protocols provide high quality DNA, suitable or sequencing, with high yield from FFPE tissue samples. Preferably, the pellet is rehydrated with a suitable buffer such as buffer BE from Covaris and more preferably a tissue lysis buffer/PK solution mix is used. The tube may be sonicated to resuspend material of the pellet, and optionally treated with RNase. A DNA purification column may be placed into a collection tube. The sample is transferred into the column and the tube spun. Following DNA purification protocol instructions, the column is washed with buffer(s) such as BW Buffer and B5 Buffer (Covaris). Finally, the column is eluted with an elution buffer, eluting the DNA from the column. The collected (eluted) DNA may be analyzed or stored long-term. Methods of the disclosure produce high quality and high yield sequencing libraries from FFPE-extracted DNA.

### Library Preparation

Having extracted DNA from a sample, methods may include library preparation, which generally includes fragmentation, adaptor ligation, and amplification. When the source is a tumor biopsy, nucleic acids in very small quantities, or preserved (e.g., FFPE) sample, extracted DNA may be fragmented via a fragmentation step that may be more gentle and less damaging than conventional protocols. In some embodiments, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. In some embodiments, it may be preferable to produce fragments that have an average size with a peak approximately within the range of about 500, preferably at least about 600 or 700, and most preferably at least about 800 base pairs (bp) to 1,000 bp. A cocktail of restriction enzymes may be composed that will, on average, cut genomic DNA on about 800 to 1,000 base intervals. Preferred embodiments use a sonicator or adaptive acoustic focusing (AFA) instrument (Covaris). Embodiments may use a Qubit instrument to evaluate quantity and/or a TAPESTATION automatic electrophoresis instrument to evaluate fragment length, using manufacturer's literature for guidelines for the sonication instrument. One approach is to shear a very small sample to the desired optical density to establish the instrument settings to be used for the bulk of the sample. The resultant shearing protocol produces 800 to 1000 base fragments.

The fragments may be repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA Repair Enzymes and Structure-specific Endonucleases are enzymes which cleave DNA at a specific DNA lesion or structure. Those enzymes can be used for repair of DNA sample degradation due to oxidative damage, UV radiation, ionizing radiation, mechanical shearing, formalin fixation (post extraction) or long-term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

In some embodiments, the end-repaired fragments are purified using magnetic beads and a magnetic separation device. A bead to DNA fragment ratio of about 0.7× may be used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. One feature of this embodiment of the disclosure is the minimal or low-bead ratio, which, in combination with the fragment length and subsequent steps, provides high quality, high-yield sequencing libraries from FFPE samples. Enzymes or other reagents may be washed away, and DNA may be eluted into a ligation mix. Methods may include ligating adaptors to the fragments to form adaptor-ligated fragments. Any suitable approach may be used. Some embodiments include dA tailing at the 3′ ends of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). The adaptor ligated fragments may be subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp.

The selected adaptor-ligated fragments may be amplified to obtain amplicons. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage. The result is a plurality of clonal amplicons copied from nucleic acid in a tumor sample.

The amplicons may have sequencing adaptors or any suitable primer binding sites at either or both ends. At this stage, a library preparation is complete.

The described extraction and library preparation protocols are optimized, compared to commercially available kits and protocols, to compensate for damage that is characteristic of FFPE samples and their extraction. For example, after emulsification of the paraffin, DNA may be subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of the steps—limited fragmentation, gentle bead clean-up, and purification after adaptor ligation with size-selection step—may contribute importantly to the preparation of high-quality sequencing libraries from FFPE samples.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline

SVs. Such an analysis may provide an identification of tumor-specific somatic SVs actually present in a patient's tumor DNA. That information is then used to design reagents to assay future samples from the patient for those same tumor-specific somatic SVs. For example, an informatics pipeline may be used to design amplification primers and fluorescent probes for the detection of such variants by a digital PCR assay. Particular embodiments identify tumor-specific SVs present in a patient's tumor DNA and then use an informatics pipeline to design primers and fluorescent hydrolysis probes useful for detecting by digital PCR those SVs in cell-free tumor DNA in blood or plasma, e.g., from a liquid biopsy.

### Sequencing

Nucleic acid obtained according to methods of the disclosure is preferably sequenced to obtain sequence data. For example, methods may include sequencing DNA from a tumor sample from the subject to obtain sequence reads.

Sequencing may be by any method known in the art. Suitable DNA sequencing techniques may include the dideoxy chain-termination sequencing technique known in the art as

Sanger sequencing, which uses labeled terminators and gel separation in a slab or capillary. Sequencing may include the sequencing by synthesis using reversibly terminated nucleotides and the detection of pyrophosphate in the technique known as pyrosequencing commercialized by ROCHE 454. Sequencing may proceed by techniques that include allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes. Sequencing may be performed using one of the single molecule, long read sequencing platforms commercialized by HELICOS, PACIFIC BIOSCIENCES, or OXFORD NANOPORE.

Sequencing techniques and instruments that may be used include, for example, those offered by ILLUMINA, INC. or ULTIMA GENOMICS. Illumina sequencing is based on the amplification of a sequencing library described above on a solid surface of a flow cell using fold-back PCR and anchored primers. Amplicons of adaptor-ligated fragments that constitute the sequencing library are annealed to oligos attached to the surface of flow cell channels that are extended by which the amplicons are bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′ terminators and fluorophores from each incorporated base are removed and the incorporation, detection and identification steps are repeated. Sequencing according to this technology is described in U.S. Pat. No. 7,960,120; U.S. Pat. No. 7,835,871; U.S. Pat. No. 7,232,656; U.S. Pat. No. 7,598,035; U.S. Pat. No. 6,911,345; U.S. Pat. No. 6,833,246; U.S. Pat. No. 6,828,100; U.S. Pat. No. 6,306,597; U.S. Pat. No. 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing generates sequence data and for short-read, ensemble sequencing platforms such as the ILLUMINA platform, the sequence data comprises a large number of short sequencing reads typically accessible from the ILLUMINA system in a computer file format known as FASTQ.

The sequencing instrument and technique relates to the biochemistry of base determination and also implicates read length and read number, with consequences for read assembly. For example, the output from Sanger sequencing on a glass-capillary instrument provided by ABI is typically a small number of medium length (several hundred bases) chromatograms that are provisionally “called” (interpreted) as bases by software and presented visually for human verification. Long read sequencing (e.g., PACIFIC BIOSCIENCES,

OXFORD NANOPORE) is meant to provide single or low numbers of much longer (>1,000) base reads. Short read sequencing (e.g., ILLUMINA) provides a large number (e.g., millions) of short reads (e.g., 50 or fewer bases) that are typically mapped to a reference and/or assembled de novo to show the original sequence. Illumina is accepted as an industry standard example of a next-generation sequencing (NGS) platform. Whatever instrument or technique is used, methods may include one or any combination of suitable “coverage” strategies, which involve determinations of what targets to sequence and at what coverage.

Coverage strategies may include, for example, transcriptome sequencing in which all RNA transcripts are sequenced redundantly, re-sequencing in which a presumptively very similar genome is known and only highly variable targets are sequenced, whole exome sequencing in which all expressed genes or exons are sequenced, or other coverage strategies. Even with a particular coverage strategy, one may opt for a certain depth of coverage. For example, for some applications, when NGS is used, 30× coverage is considered a standard coverage in which substantially all bases are sequenced redundantly such that each base, on average, appears in about 30 unique sequence reads. Certain preferred embodiments of the invention use low-pass whole genome sequencing (as used herein, “whole genome sequencing” means that a substantial portion such as at least 80% or 90% of a genome or at least a chromosome is sequenced). Low-pass whole genome sequencing (LP-WGS) is a technique in which each base in the entire genome is sequenced a few times (known as low-depth coverage) e.g., with a depth of coverage below about 5 and as low as 0.1-1 times. By reducing the depth of coverage, the cost of sequencing the whole genome is reduced while maintaining genome-scale coverage. LP-WGS is described in Christodoulou, 2023, Combined low-pass whole genome and targeted sequencing in liquid biopsies for pediatric solid tumors, NPJ Precision Onc 7:21 and Zheng, 2022, Experience of low-pass whole genome sequencing-based copy number variant analysis, Diagnostics (Basel) 12(5):1098, both incorporated by reference.

Certain preferred embodiments of the invention use whole genome sequencing (WGS). In these preferred embodiments, WGS is performed to achieve an average depth of coverage of approximately 15× (e.g., a range such as about 10× to about 20× may be employed).

This moderate depth of coverage (i.e., about 15×) provides balance between the detection of relevant genomic features—e.g., copy number variations (CNVs), structural variants (SVs), and characteristics of extrachromosomal DNA (ecDNA)—and sequencing costs, compared to very high-depth WGS (e.g., 30×, 60×, or higher). Genome-wide sequencing at approximately 15× depth allows for reliable identification and characterization of such genomic alterations across the genome and is suitable for the applications disclosed herein e.g., for sensitive tumor detection and monitoring minimal residual disease.

Whatever technique and coverage is employed, methods include sequencing nucleic acid from a tumor. In certain preferred embodiments, LP-WGS is used to sequence substantially at least about 90% of a tumor genome at a coverage of about 5× or lower. The sequencing provides sequence data of the tumor nucleic acids. The sequence data may be analyzed to create a personalized tumor mutation profile, which includes any potential tumor variants and/or mutations.

A variety of different variants and mutations may be tracked using the tumor mutation profile. Typically, these variants are structural variants. Structural variants (SVs) are genomic abnormalities that may amplify, delete, or rearrange genomic regions of a tumor. It is possible and, in fact, common for more than one SV to occur in the same tumor. As used herein, an SV generally refers to a rearrangement, duplication, or deletion of a segment of length of at least about 50 bases. Methods of the disclosure may also be used to detect tumor-specific polymorphisms and/or small indels.

### Detection of Tumor-Specific Variants

The disclosure includes methods for analyzing sequence reads, as may be obtained from nucleic acid from tumors, to identify structural variants (SVs), and optionally filter out any putative structural variants that are not somatic (e.g., germline SVs or artifacts from sample processing or sequencing) to identify SVs that are specific to the tumor, i.e., tumor variants. Methods may include comparing tumor sequence to a reference by one or more algorithms, identifying structural variants in the tumor nucleic acid, and designing primers to specifically amplify those tumor variants. Sequence reads from tumor nucleic acid may first be cleaned up, mapped to a reference, and or subject to computational workflows to detect SVs.

Reads can be cleaned using known software methods such as fastp as described in Chen, et al., 2018, fastp: an ultra-fast all-in-one FASTQ preprocessor, Bioinformatics, 34(17):1884-1890, incorporated by reference. Cleaning may include trimming adapter sequences, removing low quality bases at the ends of reads and artifacts such as polyG tails. In some FFPE embodiments cleaning may include removing reads shorter than 30 bp instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated by reference. Sequence assembly can be done by methods known in the art including reference-based assemblies, de novo assemblies, assembly by alignment, or combination methods. Sequence assembly is described in U.S. Pat. No. 8,165,821; U.S. Pat. No. 7,809,509; U.S. Pat. No. 6,223,128; U.S. Pub. 2011/0257889; and U.S. Pub. 2009/0318310, each incorporated by reference. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (sec, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. Sec McKenna et al., 2010, The Genome Analysis Toolkit: a MapReduce framework for analyzing next-generation DNA sequencing data, Genome Res 20(9):1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

A workflow for SV detection from sequence reads and for primer design may be automated using tools such as Snakemake or Nextflow and custom programming using R or Python, for example, to link input/output across the various workflow steps. Some embodiments employ a computational pipeline that uses two or more different algorithms, each intended for finding SVs, to call putative SVs and merge the results. The computational pipeline may be used for mapping reads to a reference by a first algorithm (in a first mapping) and also by a second algorithm to identify SVs by each algorithm and then selecting the better result or merging the results of the multiple mapping steps to describe the structural variants. One of the algorithms may be a graph-based algorithm. In preferred embodiments, the first algorithm adds the reads to a genomic graph and finds a path through the graph best supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27(12):2050-2060 and Cameron, 2021, GRIDSS2: comprehensive characterization of somatic structural variation using single breakend variants structural variant phasing, Genome Biol 22 (1):202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read pairs to a reference and searches for genomic regions in the reference where a significant number of read pairs align to the reference in positions anomalous with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9):677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7(8):1034-1047, incorporated herein by reference in its entirety. SplitSeq can be used to reconstruct the exact fusion sequence based on split reads and read pairs with one unmapped mate. Discordant reads can be re-aligned to reduce false positive SV calls. After merging of the SV calling paths using the first and second algorithms, the putative SVs can be annotated with genes that overlap SV breakpoints,

Methods may include filtering SVs that were identified by the mapping workflows to remove germline SVs and/or sample handling artefacts, thereby providing a set of somatic SVs, or tumor variants, present in the tumor DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, all SVs found by sequencing are preferably filtered to remove benign germline variants from the putative set, leaving a set of tumor-specific somatic SVs. Filtering may include comparing to a database of known SVs to remove from consideration those that are documented to be benign. Such a database may include the Genome Aggregation Database (gnomAD) described in Chen, 2023, A genomic mutational constraint map using variation in 76,156 human genomes, Nature 625:92-100, incorporated by reference; Genome in a Bottle SVs described in Chapman, et al., 2020, A crowdsourced set of curated structural variants for the human genome, PLOS Comp Bio, 16 (6): e 1007933, incorporated by reference; or the database of human structural variation known as dbVar described in Lappalainen. 2013, DbVar and DGVa: public archives for genomic structural variation, Nucleic Acids Res 41(Databse Issue):D936-41, incorporated by reference.

The described workflows provide for mapping the sequence reads to a reference and identifying read mappings that indicate a structural variant in the tumor nucleic acid, relative to the non-tumor nucleic acid of the subject. That structural variant is tumor specific. It is a variant specific to the tumor, herein referred to as a tumor variant. Using methods of the disclosure, the tumor variant is found by sequencing tumor nucleic acid and analyzing the sequence data. A feature of the disclosure is that such a tumor variant is confirmed by orthogonal testing. Thus, the invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### Ranking SVs

Methods of the disclosure include a step of selecting a tumor variant with an amplified copy number (relative to other tumor variants from the same tumor) as a marker variant. Preferably, the tumor variants are structural variants. The tumor variants may be detected by a computer system executing, for example, the discussed analysis software, optionally linked together in a bioinformatics pipeline. Such analysis pipelines are known in the art and generally refer to sequences of software modules that each perform at least one specific function (e.g., read cleanup, de novo assembly, file format conversion, alignment, variant calling, copy number determination, variant selection, primer design, etc.) and it is known in the art that such pipelines can be implemented by various approaches. One approach to implementing an analysis pipeline is to configure each module in a suitable computer environment and to also use shell scripts or a similar framework (Perl, BioPerl, Ruby on Rails, Python, etc.) to link the modules together. Another approach is to use an analytic platform that offers those modules and gives users ability to link together pipelines, in some cases via a graphical user interface, such as the analytic platform known as the Basespace sequence hub offered by Illumina, Inc.

Using such a computer system, the invention provides methods for ranking structural variants (SVs) and/or otherwise detecting and assigning relative ranks, in terms of clinical diagnostic utility, to a plurality of tumor specific biomarkers, such as tumor-specific variants in tumor nucleic acid. Systematically ranking SVs provides an approach for the automatic selection of which SVs to interrogate in a diagnostic assay, such as a digital PCR assay for circulating-tumor DNA in blood or plasma. Methods include analyzing sequence data from tumor nucleic acid from a tumor of a subject to identify the presence and copy numbers of a plurality of tumor-specific structural variants (SVs) in the tumor nucleic acid compared to non-tumor nucleic acid from the subject; ranking the SVs wherein higher ranks are correlated to higher copy numbers; and providing reagents for an assay that detects a tumor signature comprising one or more of the SVs selected for having the higher ranks. Thus, methods may include determining copy number of detected SVs.

Copy-number calling can then be performed to, for example, estimate tumor cell content in the sample and the degree to which the tumor genome may be rearranged. Genome-wide copy number information can be used later for prioritizing SVs for validation. Exemplary copy-number analysis can include ichorCNA described in Adalsteinsson, et al., 2017, Scalable whole-exome sequencing of cell-free DNA reveals high concordance with metastatic tumors, Nature Communications volume 8, Article number: 1324, incorporated herein by reference in its entirety. In fact, the Illumina basespace platform for analytical pipelines offers multiple CNV callers from which a user may choose including, for example, OncoCNV caller and DRAGEN CNV. Having determined copy number for a plurality of tumor variants, the computer system may be used to rank the SVs by copy number such that rank correlated to copy number, after which one or more marker variants can be selected from among the relatively higher copy number tumor SVs.

Other criteria may also be used within the ranking SV step. For example, ranking may also include assigning a high rank to a truncal SV identified as an initiating truncal mutation of the tumor. The ranking step may include application of any other suitable criteria, such as the requirement for suitable primer binding sites by which one primer pair could amplify the multiple loci or instances of duplication of the marker variant. In certain tumor signature embodiments, the computer system is used to design multiple primer pairs that are useful to detect two or more of the SVs with higher ranks as a patient-specific, tumor-specific signature of the tumor in the subject.

### Assay Design

Methods may include designing and providing a plurality of copies of a primer pair that specifically amplify the sequence and storing the plurality of copies of a primer pair as reagents for use in one or more future assays for minimal residual disease. Designing the primer pair(s) may be implemented by a computer system. The computer system may also be used to design any or all other aspects of detection assay for the marker variant(s). The computer system may be used to design, for example, suitable fluorescent hydrolysis probes such as the probes sold under the trademark TAQMAN by Thermo Fisher Scientific (Waltham, MA). Based on the copy number of a marker variant, the computer system may be programmed to calculate and store conditions for a digital PCR (dPCR) assay such as: sample volume, dilution factor, partition number, reagent concentration, instrument settings, excitation and detection wavelengths, others, or any combination thereof. Output parameters from the computer system may be used as inputs to a suitable dPCR instrument or system, optionally carried from the assay design pipeline to the dPCR system by a direct data connection (e.g., WiFi or LAN) or by a managed system such as a laboratory information management system (LIMS).

From that, the computer system may thus provide conditions for an amplification reaction that will a plurality of primer pairs designed to amplify a respective plurality of structural variants (SVs), wherein each SV of the plurality of SVs has been shown to exhibit copy amplification in the tumor nucleic acid compared to the non-tumor nucleic acid from the subject. The computer system output may also specific reagents such as the primer pairs that will amplify copies of the one or more SVs. Oligonucleotide reagents (primer pairs and fluorescent probes) maybe synthesized or obtained from a vendor such as Integrated DNA Technologies (Corralville, IA) and transferred (i.e., pipetted or dispensed) into reservoirs of the dPCR system. For example, he plurality of primer pairs are obtained as a reagent in one or more containers such as reagent tubes that are provisionally stored (e.g., lyophilized or in a freezer) and separately, subsequently dispensed to the detection assay instrument for use in the amplification reaction for detection of the plurality of SVs as a tumor-specific, patient specific signature of presences of the tumor.

Detection assay

Methods herein may include obtaining sequence data for tumor nucleic acid from a tumor from a subject; analyzing the sequence data to identify a plurality of tumor-specific variants that are in the tumor nucleic acid and that are not in non-tumor nucleic acid of the subject; selecting, from among the plurality of tumor-specific variants, a marker variant that appears duplicated in tumor nucleic acid, compared to non-tumor nucleic acid, a greater number of times than other ones of the plurality of tumor-specific variants; performing an assay to detect the marker variant in a sample from the subject; and reporting the presence of the tumor in the subject when the assay is positive for the marker variant in the sample.

The disclosed methods are useful for detection of any suitable target of interest in a sample. For example, methods are useful to detect nucleic acid from a pathogen in a mixed environmental sample or in a clinical sample that includes abundant host nucleic acid. The method may be used to detect fetal DNA in maternal blood or plasma. In certain preferred embodiments, the method is useful to detect a variant associated with a disease, such as an SV from tumor DNA, in cell-free DNA in a sample from a patient.

Any suitable sample may be used. For example, the sample may be blood, saliva, solid tissue, fine needle aspirate, a tumor biopsy (including material liberated from a formalin-fixed, paraffin embedded tumor sample), oral (e.g., buccal) swab, urine, stool, or any other sample. In certain embodiments, the sample comprises blood or plasma and the nucleic acid comprises cell-free DNA (cfDNA) in the blood or plasma.

Nucleic acids collected from biological samples may vary. These include cfDNA (cell-free DNA), ecDNA (extrachromosomal DNA), and mitochondrial cfDNA, each offering different insights and applications in diagnostics and research. cfDNA fragments can originate from various sources, including normal cell turnover, fetal DNA in maternal blood, or tumor DNA in cancer patients. cfDNA may be used for non-invasive prenatal testing, cancer detection, and monitoring minimal residual disease (MRD). Extrachromosomal DNA exists outside the chromosomes in the nucleus. It may be found in cancer cells and may be associated with oncogene amplification. Detecting ecDNA may provide information about the genetic landscape of tumors and aid in the development of targeted therapies. Mitochondrial DNA is found in the mitochondria, the energy-producing organelles in cells. Unlike nuclear DNA, mtDNA is inherited maternally and remains relatively stable across generations. Mutations in mtDNA are linked to various mitochondrial disorders and may also serve as biomarkers for certain types of cancers and neurodegenerative diseases. Beyond cfDNA, ecDNA, and mtDNA, other nucleic acid types include messenger RNA (mRNA), transfer RNA (tRNA), and microRNA (miRNA).

mRNA carries genetic information from DNA to the ribosomes, where proteins are synthesized. tRNA helps decode mRNA sequences into proteins. miRNA regulates gene expression and is involved in various cellular processes, including development, differentiation, and apoptosis.

Methods of the invention may be used to detect any nucleic acid feature of interest including, for example, specific sequences, genes, or variants (e.g., mutations), which may include polymorphisms, small indels, or structural variants (which may include deletions, rearrangements, large indels, translocations, copy number variants, or others). In certain embodiments, the variant is a structural variant (SV) and a pre-amplification is performed with a PCR primer pair design to anneal to sites that flank a breakpoint of the SV. Preferred embodiments provide methods useful to detect nucleic acid fragments containing targeted structural variants present in a sample at low abundance (e.g., as low as one copy).

In preferred liquid biopsy and dPCR for MRD embodiments, the obtaining step may involve receiving a blood collection tube or container containing blood or plasma that was obtained from the subject via blood draw. The sample may include cell-free DNA from blood or plasma from the subject. The sample may be less than about 100 mL of blood or plasma and wherein the cell-free DNA is present at a concentration between about 1 and 50 ng/ml or lower in the blood or plasma circulating in the subject. Any given unduplicated locus in ctDNA may be present only at a very low concentration such that any given target or locus is statistically likely to be beyond/below the limit of detection.

In certain optional in vitro “pre amplification” embodiment, the detection may proceed by at least two distinct stages or mechanisms that include (copying the marker variant using variant-specific primers and tailed primers to form tailed amplicons and amplifying the tailed amplicons in the presence of probes that indicate the presence of amplicons from the target of interest in the aqueous compartment. In some embodiments, a pre-amplification step may use primers designed to specifically amplify the marker variant. For example, if the variant is a structural variant, the pre-amplification may use a pair of primers designed to anneal to nucleic acid at locations that flank a breakpoint of the structural variant. This strategy further enriches the sample for copies of the marker variant, ensuring that the presence of the variant is detected in the subsequent detection steps. This pre-amplification step specifically addresses problems associated with some dead volume of sample that resists detection by existing digital PCR (dPCR) approaches. Due to the stochastic nature of sampling, some very minor fraction of a sample will, by chance, typically go undetected by dPCR. Here, the pre-amplification step may increase quantity of the marker variant prior to the partitioning and dPCR detection, reducing the likelihood that the target of interest will be undetected due to stochastic loss in dead volume. After the pre-amplification, the sample may be partitioned into aqueous compartments.

Regardless of any optional in vitro “pre-amplification” embodiments, the detection assay is useful to detect the tumor, and may be used as an MRD assay, even when any unduplicated tumor locus would otherwise be beyond the LoD for the assay.

### Limit of detection

Tumor detection assays of the invention take advantage of in vivo preamplification of certain tumor targets. Methods may be used in assays for minimal residual disease (MRD), e.g., after a treatment to eradicate the tumor. Methods my include obtaining a sample from a subject who has undergone treatment for a tumor; performing an amplification reaction in the sample using a primer pair that is designed to amplify a tumor-specific marker variant that appears duplicated in tumor nucleic acid from the tumor, compared to non-tumor nucleic acid from the subject, a greater number of times than other tumor-specific variants that have been shown to be present in the tumor nucleic acid; and reporting the residual presence of the tumor after the treatment when the primer pair generates amplicons by the amplification reaction. The sample may include less than about 100 mL of the blood or 0.1 ng to 5 mL of the plasma and the cell-free DNA may be present at a concentration between about 1 and 50 ng/ml or lower in the blood or plasma circulating in the subject. Under such conditions, it may be statistically improbable to detect any given ctDNA fragment from a conventional liquid biopsy blood draw. Methods of the invention break that statistical LoD using an analytical workflow that includes analyzing sequence data from tumor nucleic acid to identify a plurality of tumor-specific variants, and then selecting one of those identified tumor variants that has an amplified copy number, in the tumor nucleic acid, relative to the others, and that is not found in healthy, non-tumor DNA of the subject. That amplified copy number tumor variant may be deemed a marker variant for purposes herein and an assay is prepared and/or performed to detect the marker variant in a sample from the subject as a test for evidence of the presence of the tumor in the subject.

In certain embodiments, in vivo amplification of tumor-specific DNA sequences occurs through the formation of extrachromosomal DNA (ecDNA). This ecDNA formation, which can arise from mechanisms such as chromothripsis, results in high copy numbers of tumor-specific variants and frequently leads to the focal amplification of oncogenes located on the ecDNA.

The present disclosure relates to methods, systems, and compositions for identifying, characterizing, and using structural variants (SVs) within extrachromosomal DNA (ecDNA) as biomarkers for the detection, monitoring, and assessment of minimal residual disease (MRD) in subjects with cancer.

The accurate detection of minimal residual disease (MRD) following cancer treatment is important for predicting patient outcomes, guiding adjuvant therapy choices, and detecting early relapse. Conventional MRD biomarkers often suffer from limitations in sensitivity, e.g., when tumor burden is very low. Extrachromosomal DNA (ecDNA) are circular, non-chromosomal DNA structures often found at very high copy numbers (e.g., 10-100s of copies per neoplastic cell) and frequently harbor oncogenes that drive tumor progression and therapeutic resistance. The amplification of ecDNA provides an in vivo signal enhancement, and their structural variants (SVs) may act as tumor biomarkers.

In many embodiments, the targeted ecDNA SVs are those that arise from the very formation of ecDNA. Large-scale DNA damage events, such as, but not limited to, chromothripsis and chromosome shattering, can produce numerous small DNA fragments. The subsequent re-ligation and circularization of these fragments to form ecDNA generate a multitude of SVs, including deletions, duplications, inversions, and rearrangements, relative to the reference germline genome. These SVs, present from the initial formation of a given ecDNA element, are referred to as “founder SVs.” Such founder SVs, if the ecDNA construct undergoes positive selection and amplification, become highly represented within the cancer cell's genomic material.

In preferred embodiments, identification of ecDNA amplicons is performed using whole-genome sequencing (WGS) data obtained from a biological sample from a subject (e.g., tumor tissue, blood, plasma, other bodily fluids). In certain embodiments, bioinformatics pipelines are employed, as conventional SV callers alone are insufficient to determine the extrachromosomal origin of an SV.

Such pipelines include, for example, algorithms (e.g., AmpliconArchitect) and other computational tools. These tools are designed to analyze WGS data for signatures e.g., of focal amplification and circularity. Other methodologies, e.g., using Circle-seq data or ATAC-seq data, may also be employed or integrated to identify or validate ecDNA structures.

Once ecDNA structures are computationally resolved, SVs identified using standard SV calling algorithms from the WGS data are mapped to these ecDNA structures. An SV is determined to be an ecDNA SV if its genomic coordinates and breakpoints fall within the boundaries of an identified and validated ecDNA amplicon. This mapping process confirms the extrachromosomal context of the SV.

Not all identified ecDNA SVs may be equally suitable for MRD detection. Embodiments of the invention include steps for prioritizing ecDNA SVs to maximize MRD assay sensitivity.

In some embodiments, ecDNA SVs are designated as “higher ranked” or preferential targets for MRD assays. This is due to several characteristics. As ecDNA elements can be present in tens to hundreds of copies per cell, founder SVs located on these elements are correspondingly amplified. This significantly increases the likelihood of detecting the SV in a sample with low tumor burden, such as cfDNA isolated from plasma. The biological processes leading to high ecDNA copy numbers serve as an in vivo signal amplification mechanism, going beyond what can be achieved by targeting single-copy or low-copy chromosomal variants. Further, the circular structure of ecDNA, when shed into biofluids as cell-free DNA (cfDNA), may confer increased resistance to exonuclease degradation compared to linear cfDNA fragments. This could result in a longer half-life and accumulation of ecDNA-derived cfDNA in the bloodstream, thereby improving the signal-to-noise ratio for MRD detection.

In certain embodiments, ecDNA SVs are assessed for their proximity to or inclusion of known oncogenes, tumor suppressor genes, or other cancer-relevant genes. The presence of key oncogenes (e.g., MYC, MYCN, EGFR, PDGFRA, MET, the MECOM-PIK3CA-SOX2 gene cluster, and the CDK4-MDM2 gene cluster, etc) within the ecDNA construct carrying the SV may indicate that the ecDNA plays a role in tumor maintenance. This assessment can be performed by annotating the genomic regions encompassed by the ecDNA with known gene locations and functions.

In some embodiments, the stability of a ecDNA SV may be assessed by analyzing its presence and relative abundance in samples taken at different time points from the subject (e.g., pre-treatment, during treatment, post-treatment) exposed to therapy. Techniques such as droplet digital PCR (ddPCR), quantitative PCR (qPCR), or targeted sequencing focusing on the ecDNA

SV can be used for such longitudinal monitoring.

Algorithms may be developed or employed to predict the stability of ecDNA constructs based on features such as size, complexity, gene content, presence of replication origins, or signatures of active maintenance.

Examining the prevalence and consistency of specific ecDNA amplicons and their SVs across different regions of a tumor or in metastatic sites can provide insights into their stability and dissemination.

ecDNA SVs may be selected or excluded for use in an MRD assay. ecDNA SVs that are confirmed to be located on highly amplified ecDNA, associated with key oncogenes, and deemed stable are considered strong candidates for MRD biomarkers. An ecDNA SV may be excluded if the ecDNA construct the SV is located on is deemed highly unstable (e.g., significant variability in copy number). This prevents false-negative MRD results due to biomarker loss rather than change in disease status.

Selected ecDNA SVs can be incorporated into various MRD assay platforms.

Assays (e.g., PCR-based, sequencing-based) are designed to detect and quantify the prioritized ecDNA SV. These assays can be applied to cfDNA extracted from liquid biopsy samples (e.g., plasma, serum, urine), offering a non-invasive means of MRD monitoring. They may also be used on tumor tissue DNA for initial biomarker discovery and validation. The levels of the ecDNA SV can be monitored over time to assess treatment response, detect early relapse, or guide therapeutic strategies. A decrease in the ecDNA SV level may indicate treatment efficacy, while an increase may signal disease recurrence or progression.

The use of ecDNA SVs as MRD biomarkers, according to the methods described herein, offers several advantages. Due to the high copy number of ecDNA, targeting ecDNA SVs can provide significantly higher sensitivity compared to single-copy chromosomal alterations. Further, ecDNA has been observed across a wide range of cancer types, suggesting broad applicability of this MRD detection strategy.

### Digital PCR

The described detection assay may be any suitable assay including, for example, nucleic acid sequencing, DNA microarray analysis, fluorescent in situ hybridization, PCR, quantitative PCR, or digital PCR (dPCR). In preferred embodiments, the detection assay is dPCR and the sample comprises blood or plasma from the subject and the assay analyzes cell-free nucleic acid in the blood or plasma. For the assay, dPCR may include partitioning the sample into aqueous partitions that include PCR reagents and fluorescent probes for the amplicons and conducting the amplification reaction in the aqueous partitions. The assay comprises performing an amplification reaction to detect amplification of the copies of the one or more SVs.

The assay includes partitioning the sample into aqueous partitions and performing an amplification reaction in the aqueous partitions using at least one primer specific for the sequence and a probe that provides a signal when the amplification reaction using at least one primer generates an amplification product. The method may include partitioning the sample into aqueous partitions that include PCR reagents and fluorescent probes for the amplicons, conducting the amplification reaction in the aqueous partitions, and detecting fluorescence from the partitions to detect the residual presence of the tumor after the treatment. Those dPCR steps may all be automated and/or performed using a commercially available dPCR instrument or system. The dPCR system may itself operate to perform the amplification reaction under conditions (sample volume, cfDNA concentration, dilution, partition size) at which an unduplicated locus in the tumor nucleic acid would be statistically beyond a limit of detection. The dPCR system may detect fluorescence from the partitions and provide output indicating a number of partitions that include the marker variant.

For a subject in whom a tumor has been diagnosed, and a sample of the tumor (biopsy, FFPE slice) obtained, a treatment may have been administered to eradicate the tumor. For example, the person may have undergone surgical resection to remove the tumor, radiation therapy to ablate the tumor, or chemotherapy to kill cells of the tumor. The person may spend some amount of time feeling the benefit of the treatment, living a cancer-free life. However, an insidious aspect of cancer is that, even after treatment to eradicate a tumor, that cancer may return, later, e.g., months or even years later. For a cancer that does return, because some vanishingly small number of cells escaped eradication by the treatment (i.e., MRD), there may still be the opportunity to kill those cells and cure the cancer if the presence of that MRD is detected in good time. In such situations, the invention provides methods that provide a detection assay that can detect MRD even when other biomarkers are expected to be beyond the LoD for the assay. Methods of the invention may be used to provide the elements of a detection assay (e.g., assay design and reagents such as PCR primers and detection probes) that may be kept and used repeatedly over time, e.g., tens of times or more, over months and years, conveniently and inexpensively. The detection assay may be a PCR-based assay that only needs a blood draw, as described, so that, after undergoing treatment, a person may know via a relatively quick, inexpensive, and minimally invasive test, whether there is any evidence of MRD.

1. A method comprising:
obtaining sequence data for tumor nucleic acid from a tumor from a subject;
analyzing the sequence data to identify a plurality of tumor-specific variants that are in the tumor nucleic acid and that are not in non-tumor nucleic acid of the subject;
selecting, from among the plurality of tumor-specific variants, a marker variant that appears duplicated in tumor nucleic acid, compared to non-tumor nucleic acid, a greater number of times than other ones of the plurality of tumor-specific variants;
performing an assay to detect the marker variant in a sample from the subject; and
reporting the presence of the tumor in the subject when the assay is positive for the marker variant in the sample.

2. The method of claim 1, wherein the assay comprises detection method under conditions at which an unduplicated locus in the tumor nucleic acid would be statistically beyond a limit of detection.

3. The method of claim 1, wherein a limit of detection is increased by increasing the number of variants within a sample

4. The method of claim 1, wherein the sample comprises blood or plasma from the subject and the assay comprises digital PCR to detect cell-free nucleic acid in the blood or plasma.

5. The method of claim 1, wherein the obtaining step comprises sequencing DNA from a tumor sample from the subject to obtain sequence reads.

6. The method of claim 5, wherein the analyzing step comprises mapping the sequence reads to a reference and identifying read mappings that indicate a structural variant in the tumor nucleic acid relative to the non-tumor nucleic acid of the subject.

7. The method of claim 5, wherein a quantitative measure of sequence reads for a structural variant is indicative of a quantity of duplications for the variant.

8. The method of claim 1, further comprising designing a primer pair useful to amplify the marker variant, wherein the assay comprises an amplification reaction.

9. The method of claim 1, wherein the sample comprises cell-free DNA from blood or plasma and the assay comprises dividing the sample into a plurality of partitions wherein at least one partition includes one fragment of the cell-free DNA that includes one copy of the marker variant that was duplicated within the tumor nucleic acid.

10. The method of claim 9, wherein, due to a quantity of the cell-free DNA circulating in the blood or plasma in the subject and due to a volume of the sample, it is mathematically more probable that (i) the cell-free DNA contains a copy of a duplicated locus than that (ii) of the unduplicated locus.

11. The method of claim 9, further comprising providing each of the plurality of partitions with PCR reagents, a primer pair useful to amplify the variant, and detectably labeled probes for an amplification product of the primer pair.

12. The method of claim 11, wherein:
the sequence data is obtained by sequencing DNA from a formalin-fixed, paraffin embedded slice of the tumor;
the partitions are aqueous droplets;
the assay is droplet digital PCR;
the detectably labeled probes are fluorescent hydrolysis probes;
detecting fluorescence from the aqueous droplets indicates the presence of the tumor nucleic acid in the sample; and/or
the assay is performed to detect minimal residual disease after the treatment.

13. A method for detecting indicia of disease, the method comprising:
identifying a duplicated sequence within tumor nucleic acid;
performing an assay to detect the sequence in a sample from a subject at conditions under which an unduplicated genomic locus of the subject is more likely to be undetectable than to be detectable; and
reporting the presence of the tumor in the subject when the sequence is detected using the assay.

14. The method of claim 13, wherein, based on a volume of the sample and a concentration of nucleic acid in the sample, it is statistically probable that the unduplicated genomic locus is not detected in the sample.

15. The method of claim 13, wherein the sample comprises blood or plasma from the subject and method comprises capturing cell-free nucleic acid from the blood or plasma and performing the assay on the cell-free nucleic acid.

16. The method of claim 13, wherein the assay includes partitioning the sample into partitions and performing an amplification reaction in the partitions using at least one primer specific for the sequence and a probe that provides a signal when the amplification reaction using at least one primer generates an amplification product.

17. The method of claim 16, wherein the partitions comprise aqueous droplets and the assay comprises droplet digital PCR with sequence-specific fluorescent hybridization probes.

18. The method of claim 13, wherein the assay is for cell-free nucleic acid, wherein the sample is less than about 100 mL of blood or plasma, wherein the cell-free nucleic acid is present at a concentration between about 0.1 and 50 ng/ml in the blood or plasma circulating in the subject, and wherein the sequence is duplicated to at least about 2 copies in a genome of the tumor.

19. The method of claim 13, wherein the identifying step comprises sequencing DNA from a formalin-fixed, paraffin embedded slice of the tumor to obtain sequence reads and mapping the sequence reads to a reference, and identifying read-mappings consistent with a structural variant that is duplicated in the tumor nucleic acid.

20. The method of claim 13, wherein the identifying step comprises sequencing DNA from the tumor to obtain sequence reads, mapping the reads to a reference to identify a plurality of tumor-specific structural variants (SVs), ranking the SVs by copy number wherein higher ranks are correlated to higher copy numbers, and selecting a high-ranking SV as the sequence.

21. The method of claim 20, wherein the assay is designed to detect two or more of the SVs with higher ranks as a patient-specific, tumor-specific signature of the tumor in the subject.

22. The method of claim 21, where the combination of duplications (copies) is used to estimate the likelihood of a positive signal.

23. The method of claim 13, further comprising designing and providing a plurality of copies of a primer pair that specifically amplify the sequence and storing the plurality of copies of a primer pair as reagents for use in one or more future assays for minimal residual disease.

24. A method comprising:
obtaining a sample from a subject who has undergone treatment for a tumor;
performing an amplification reaction in the sample using a primer pair that is designed to amplify a tumor-specific marker variant that appears duplicated in tumor nucleic acid from the tumor, a greater number of times than other tumor-specific variants that have been shown to be present in the tumor nucleic acid; and
reporting the residual presence of the tumor after the treatment when the primer pair generates amplicons by the amplification reaction.

25. The method of claim 3, wherein the obtaining step includes receiving a blood collection tube or container containing blood or plasma that was obtained from the subject via blood draw.

26. The method of claim 24, wherein the sample comprises cell-free DNA from blood or plasma from the subject.

27. The method of claim 25, wherein the sample is less than about 100 mL of blood or plasma and wherein the cell-free DNA is present at a concentration between about 0.1 and 50 ng/mL in the blood or plasma circulating in the subject.

28. The method of claim 26, wherein under conditions of the amplification reaction it is more probable that an unduplicated genomic locus from the tumor would not encounter the primer pair than that the duplicated genomic locus would encounter the primer pair.

29. The method of claim 25, further comprising partitioning the sample into aqueous partitions that include PCR reagents and fluorescent probes for the amplicons and conducting the amplification reaction in the aqueous partitions.

30. The method of claim 29, further comprising detecting fluorescence from the partitions to detect the residual presence of the tumor.

31. The method of claim 24, wherein the amplification reaction uses a plurality of primer pairs designed to amplify a respective plurality of structural variants (SVs), wherein members of the plurality of SVs have been shown to exhibit copy amplification in the tumor nucleic acid compared to the non-tumor nucleic acid from the subject.

32. The method of claim 31, wherein the plurality of primer pairs are provided as a reagent in one or more containers for use in the amplification reaction for detection of the plurality of SVs as a tumor-specific, patient specific signature of presences of the tumor.

33. The method of claim 32, wherein the plurality of SVs are detected in multiplex in the one amplification reaction using a respective plurality of detectably labeled probes.

34. A method comprising:
analyzing sequence data from tumor nucleic acid from a tumor of a subject to identify the presence and copy numbers of a plurality of tumor-specific structural variants (SVs) in the tumor nucleic acid compared to non-tumor nucleic acid from the subject;
ranking the SVs wherein higher ranks are correlated to higher copy numbers; and
providing reagents for an assay that detects a tumor signature comprising one or more of the SVs selected for having the higher ranks.

35. The method of claim 34, wherein the reagents comprise primer pairs that amplify copies of the one or more SVs.

36. The method of claim 34, further comprising performing the assay on a sample from a subject to detect the tumor in the subject by detecting copies of the one or more SVs.

37. The method of claim 36, wherein the copies are detected in cell free DNA from blood or plasma in the sample.

38. The method of claim 37, wherein the sample include less than about 100 mL of the blood or plasma and wherein the cell-free DNA is present at a concentration between about 0.1 and 50 ng/mL in the blood or plasma circulating in the subject.

39. The method of claim 38, wherein the assay comprises performing an amplification reaction to detect amplification of the copies of the one or more SVs.

40. The method of claim 26, wherein under conditions of the amplification reaction it is more probable that an unduplicated genomic locus from the tumor would not be present in the sample than that the duplicated genomic locus would be present in the sample.

41. The method of claim 39, further comprising:
partitioning the sample into aqueous partitions that include PCR reagents and fluorescent probes for the amplicons;
conducting the amplification reaction in the aqueous partitions; and
detecting fluorescence from the partitions to detect the residual presence of the tumor after the treatment.

42. The method of claim 34, wherein the ranking step further includes assigning a high rank to a truncal SV identified as an initiating truncal mutation of the tumor.

43. A method comprising:
obtaining sequence data for tumor nucleic acid from a tumor from a subject;
analyzing the sequence data to identify a plurality of tumor-specific variants that are in the tumor nucleic acid and that are not in non-tumor nucleic acid of the subject;
selecting, from among the plurality of tumor-specific variants, a variant that will statistically be present in a blood sample at least 2× above the average number of variants in the sample;
performing an assay to detect the marker variant in a sample from the subject; and
reporting the presence of the tumor in the subject when the assay is positive for the marker variant in the sample.

44. The method of claim 5, wherein the plurality of tumor-specific variants includes tumor-specific variants within extra-chromosomal DNA (ecDNA), and the marker variant is a tumor-specific variant within the ecDNA.

45. The method of claim 20, wherein the plurality of tumor-specific SVs include SVs within ecDNA,

46. The method of claim 45, wherein the higher ranks are further correlated to being within ecDNA.
